(19) United States
(12) Patent Application Publication
(10) Pub. No.: US 2019/0242864 A1
(43) Pub. Date: Aug. 8, 2019

(54) WATER QUALITY SENSING

(71) Applicant: RS HYDRO LIMITED, Stoke Prior Bromsgrove Worcestershire (GB)

(72) Inventors: Robert STEVENS, Stoke Prior Bromsgrove Worcestershire (GB); Kieran KHAMIS, Stoke Prior Bromsgrove Worcestershire (GB)

(73) Assignee: RS HYDRO LIMITED, Stoke Prior Bromsgrove Worcestershire (GB)

(21) Appl. No.: 16/327,821

(22) PCT Filed: Aug. 24, 2017
(86) PCT No.: PCT/GB2017/052496

§ 371 Date: Feb. 23, 2019

(30) Foreign Application Priority Data
Aug. 25, 2016 (GB) 1614497.4

(51) Int. Cl. G01N 33/18; G01N 21/15; G01N 21/53; G01N 21/64; G01N 21/85

(52) U.S. Cl. CPC G01N 33/1806; G01N 21/15; G01N 21/85; G01N 21/6486; G01N 21/53

(57) ABSTRACT

A water quality sensor comprising a fluorescence sensor arrangement 16 operable to measure a fluorescence intensity, a temperature sensor 22 and a turbidity sensor 24, the outputs of which are used in combination to derive a value for the BOD concentration of a sample, wherein the output of the temperature sensor 22 is used to apply a correction to the sensed fluorescence intensity value using the equation

$${F}_{ref} = \frac{{F}_{mes}}{1 + ρ ( {T}_{mes} - {T}_{ref} )}$$

where F is the fluorescence intensity signal, T is temperature (° C.), and the subscripts mes and ref represent the measured and reference values respectively, and ρ is a temperature compensation coefficient.

### CROSS-REFERENCE TO RELATED APPLICATION

This application is the U.S. national phase of PCT Application No. PCT/GB2017/052496 filed on Aug. 24, 2017, which claims priority to GB Patent Application No. 1614497.4 filed on Aug. 25, 2016, the disclosures of which are incorporated in their entirety by reference herein.

### TECHNICAL FIELD

This invention relates to a water quality sensor, and in particular to a sensor suitable for use in providing information, substantially in real time, regarding the quality of water, and to a method of use thereof. In particular the invention relates to a sensor operable to sense the biological oxygen demand (BOD) concentration in water. It may be applied to a flow of water, or to standing water.

### BACKGROUND

Where waste water is treated in a water treatment plant prior discharge into a water course, it is important that the treated water is of sufficiently good quality to ensure that the likelihood of damage to the natural environment, of which the water course forms, part is minimised.

Failures in the operation of a treatment plant can result in the water discharged therefrom failing to meet sufficiently high standards and the health of wildlife, for example fish, in or close to the water course is put at risk. Where incidents have occurred resulting in the discharged water being of poor quality or in the death of fish or other wildlife, then the owners of the treatment plant may be subject to fines or other penalties.

One parameter that is used in determining whether or not discharged water meets the required quality is the BOD concentration as this provides an indication of the quantity of reactive dissolved organic matter in the water.

Typically, BOD is measured under laboratory conditions using a 5-day test. Whilst such tests provide an indication of the BOD concentration of a sample, the length of time taken for the test results to issue is sufficient that a significant quantity of water may have been discharged into a water course during the time taken for the test to be completed.

If the result of the test indicates that the BOD concentration is inappropriate for discharge, whilst further discharge may be avoided, a significant quantity of water that fails to comply with the required BOD concentrations will already have been discharged, with the result that the health of wildlife, etc, may have already been put at risk.

### SUMMARY

It is an object of the invention to provide a sensor whereby water quality of a test sample can be determined substantially in real time.

According to the present invention there is provided a water quality sensor comprising a fluorescence sensor arrangement, a temperature sensor and a turbidity sensor, the outputs of which are used in combination to derive a value for the BOD concentration of a sample, wherein the output of the temperature sensor is used to apply a correction to the sensed fluorescence signal value using the equation

$${F}_{ref} = \frac{{F}_{mes}}{1 + ρ ( {T}_{mes} - {T}_{ref} )}$$

where F is the fluorescence signal, T is temperature (° C.), and the subscripts mes and ref represent the measured and reference values respectively, and ρ is a temperature compensation coefficient.

It has been found that the irradiation of the sample with an appropriate energisation or excitation electromagnetic radiation leads to fluorescence of certain molecules contained within a sample, the magnitude of which is related to the level of BOD within the sample. However, the relationship between the fluorescence and the BOD concentration also depends upon other factors including the temperature and the turbidity of the sample. Failure to take these factors into account reduces the measurement accuracy. Accordingly, by measuring these parameter values as well as the fluorescence, and correcting the fluorescence reading to take into account the sensed temperature and turbidity levels, a good indication or approximation of the BOD concentration within the sample under test can be obtained.

The fluorescence sensor arrangement is preferably arranged to irradiate the sample with light of wavelength in the region of 260-300 nm, and to detect fluorescence of wavelength in the region of 295-405 nm, such an arrangement being sensitive to BOD concentrations within the sample.

By using the fluorescence, corrected to take into account the effects of temperature and turbidity, as an indicator of the BOD concentration within a sample, it will be appreciated that results indicative of the concentration of BOD within a sample are available very quickly, substantially in real time. Accordingly, if the sensor is used at a water treatment plant to conduct tests on the water about to be discharged into a natural water course, in the event that it is detector by the sensor that the BOD concentration falls outside of an acceptable range, then an appropriate alarm can be activated to allow operators to modify the operation of the plant to avoid the ongoing discharge of water with inappropriate BOD concentrations from the plant. By way of example, discharge of water from the plant may be temporarily interrupted to prevent water that fails to meet the required standards from being discharged therefrom.

The invention further relates to a water testing method comprising the steps of using a fluorescence sensor arrangement to measure a fluorescence of a sample in response to irradiation of the sample with electromagnetic radiation of an excitation wavelength, measuring the temperature and turbidity of the sample, using the measured temperature and turbidity to derive a corrected fluorescence intensity, and using the corrected fluorescence intensity to derive an indication of the BOD concentration of the sample, wherein the measured temperature is used to apply a correction to the sensed fluorescence intensity value using the equation

$${F}_{ref} = \frac{{F}_{mes}}{1 + ρ ( {T}_{mes} - {T}_{ref} )}$$

where F is the fluorescence signal, T is temperature (° C.), and the subscripts mes and ref represent the measured and reference values respectively, and ρ is a temperature compensation coefficient.

### BRIEF DESCRIPTION OF THE DRAWINGS

The invention will further be described, by way of example, with reference to the accompanying drawings, in which:

FIG. 1a is a schematic diagram illustrating a sensor in accordance with an embodiment of the invention;

FIG. 1b is an enlargement of part of FIG. 1a; and

FIG. 2 is a scatter plot displaying the relationship between corrected fluorescence measurements and the BOD concentration of a number of independent samples.

### DETAILED DESCRIPTION

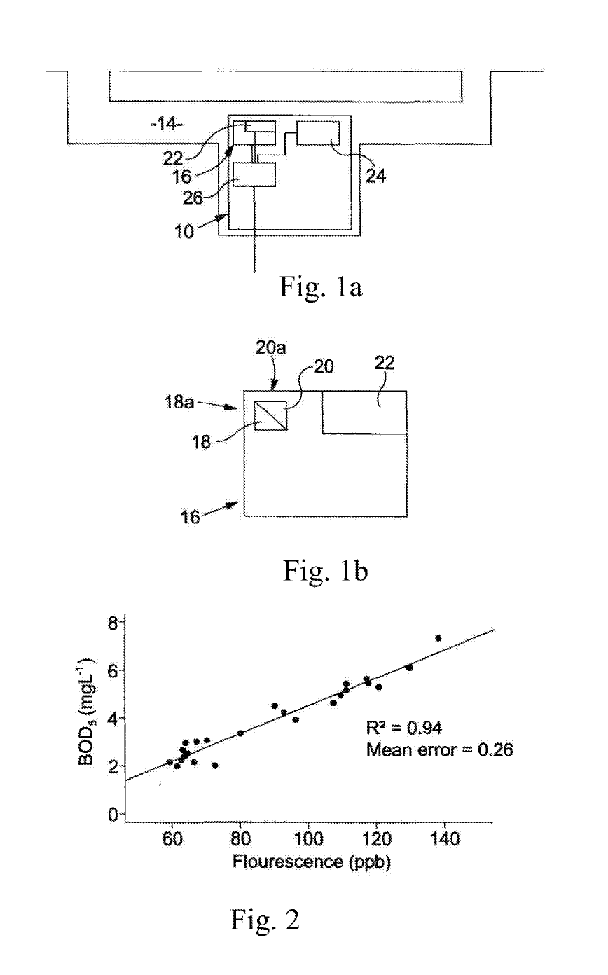

Referring to the accompanying drawings, a sensor 10 is illustrated, the sensor 10 being operable to sense the BOD concentration within a water sample. Whilst the sensor of the invention may be employed in a range of applications, in FIG. 1a the sensor 10 is employed in such a manner that water about to be discharged from a waste water treatment plant along a line 12 is tested. As illustrated, a test line 14 runs in parallel to part of the line 12, and the sensor 10 is operable to undertake tests upon sample of water passing along the test line 14. The sensor 10 may be fully immersed within the test line 14. Alternatively, just a sensor probe thereof may be immersed, if desired. In other arrangements, the sensor 10 may be deployed directly within the line 12. It will be appreciated that this represents just one application in which the sensor 10 may be used, and the invention is not restricted in this regard.

The sensor 10 includes three test modules. Firstly, it includes a fluorescence sensor arrangement 16. The fluorescence sensor arrangement 16 includes a light source 18, for example in the form of an array of LEDs or the like, operable to irradiate the sample under test, through a transparent window 18a, with electromagnetic radiation of an excitation wavelength falling within the range of 260-300 nm. By way of example, the excitation frequency may be of wavelength approximately 285 nm (say, ±10 nm). It has been found that a number of free amino acids, peptides and proteins and dissolved organic matter molecules that are typically present in human or animal waste, when irradiated with light of this frequency, will fluoresce resulting in the output of a detectable fluorescence in the wavelength range of 295-405 nm. In order to detect this fluorescence, the sensor arrangement 16 further includes a light sensor 20, for example in the form of a suitable photo detector, arranged to detect such fluorescence via a transparent window 20a. The photodetector is conveniently operable to detect fluorescence of wavelength in the region of 350 nm (say, ±55 nm). The light sensor 20 and light source 18 are conveniently angled to one another relative to the sample under test. Preferably, they are arranged perpendicularly to one another such that the sensor 20 only detects fluorescence rather than having the output from the light source 18 directly incident thereon. However, other configurations are possible without departing from the scope of the invention.

The arrangement 16 could comprise, for example, a Pyrex flow cell housed in a stainless steel case with optical components installed along two orthogonal axes. The instrumentation components of such an arrangement may include: (i) an excitation branch (LED, filter and condenser lens) and, perpendicular to this, (ii) a detection branch (lens, filter and photodiode). It will be appreciated, however, that this represents merely one option, and that the arrangement 16 may in practise differ substantially from this without departing from the scope of the invention. The LEDs, filters and photodiodes, or other similar components, are selected to match the spectral properties of the fluorescence peak of interest. For peak fluorescence, the selected excitation wavelength (285 nm) differed from the commonly reported maximal excitation for peak which is generally taken to be in the range of 270-280 nm. The wavelength was chosen due to: (i) previous research on urban river systems highlighting the importance (in urban rivers) of the peak at 282±3 nm; (ii) high current draw, low optical output and decreased lifetime of lower wavelength ultraviolet (UV) LEDs, and; (iii) comparability with other in-situ fluorimeters.

The second test module takes the form of a temperature sensor 22, for example in the form of a thermistor, conveniently integrated with the arrangement 16. The thermistor is preferably of good accuracy, for example having a sensitivity in the region of 0.01° C. The thermistor may be attached to the inside of the Pyrex cell, and so be operable to monitor temperature of the sample enabling the quantification of thermal quenching of the fluorescence signal and thereby allowing its subsequent correction.

Thirdly, the sensor 10 includes a turbidity sensor 24 operable to detect or sense the turbidity of the sample under test.

Whilst not illustrated, as certain of the modules of the sensor 10 are optically based, the sensor housing conveniently includes a cleaning mechanism, for example in the form of a wiper, operably to clean the windows 18a, 20a through which measurements are taken. It will be appreciated that the provision of the wiper enhances the accuracy with which measurements can be made through avoiding the build-up of debris upon the window. Whilst a wiper represents one form of cleaning arrangement, it will be appreciated that other forms of cleaning arrangement may be provided. For example, arrangements in which a jet of clean water or a suitable gas are directed periodically over the window to dislodge debris therefrom may be used. Indeed, depending upon the application in which the sensor 10 is used, there may be no requirement to provide any form of cleaning arrangement.

It has been found that by making corrections to the measured fluorescence levels detected using the arrangement 16 to correct for temperature variations and turbidity variations, a substantially linear relationship between the corrected fluorescence levels and the BOD concentration of the sample exists. As shown in FIG. 1a, the sensor 10 includes a control unit 26 receiving the outputs of the sensor modules and operable to correct the output of the arrangement 16 to take into account the sensed temperature and turbidity, and to output a signal, using the corrected fluorescence value, indicative of the sensed BOD concentration. The output signal may be transmitted wirelessly, if desired, or output via a cabled connection.

FIG. 2 illustrates some experimental results indicating the relationship between the corrected fluorescence and the BOD concentration (expressed in mg/l) present in a range of samples, from which it is clear that the relationship is substantially linear. Accordingly, using the outputs of the temperature and turbidity sensors 22, 24 to apply an appropriate corrections to the fluorescence detected by the arrangement 16 to derive a corrected fluorescence level, an indication of the BOD concentration of the water sample under test can be derived and output by the control unit 26.

Temperature correction may be achieved using the formula:

$${F}_{ref} = \frac{{F}_{mes}}{1 + ρ ( {T}_{mes} - {T}_{ref} )}$$

where F is the fluorescence signal T is temperature (° C.) and subscripts mes and ref represent the measured and reference values respectively, and ρ is the temperature compensation coefficient and is calculated by creating a regression of temperature vs fluorescence intensity for the molecules of interest, and then calculating the ratio of the slope to the intercept of that regression.

Turbidity correction may be achieved using coefficients derived from an empirical regression model that includes the terms turbidity, fluorescence intensity and interactions between turbidity and fluorescence intensity. Where the mean particle size in the sample is known, for example in water treatment works or controlled industrial processes, it can be included in the model to optimise the data correction procedure. In natural river systems, wherein the sediment particle size is unknown, a site specific model can be developed using sediments collected from the river system of interest.

Whilst in the description hereinbefore the fluorescence effect that is relied upon to derive a BOD concentration value for the sample relates to fluorescence in the 350 nm region arising through the application of an excitation irradiation of wavelength in the 285 nm region, it will be appreciated that the invention is not restricted in this regard, and that other fluorescence peaks may be used. By way of example, fluorescence in the region of 365 nm in response to an excitation emission in the region of 490 nm may be used. Of course, the sensor may be operable to detect BOD concentrations whilst also operable to detect DOC concentrations and/or the presence of other materials in the sample under test.

As described hereinbefore, the invention is advantageous in that water quality tests may be undertaken and produce results substantially in real time. Accordingly, the test results may be used to monitor the operation of, for example, a waste water treatment plant, and to allow action to be taken in the event that the sensor detects that the water quality is failing to meet predetermined conditions.

Whilst a specific embodiment of the invention is described hereinbefore, a number of modifications and alterations may be made thereto without departing from the scope of the invention as defined by the appended claims.

1. A water quality sensor comprising a fluorescence sensor arrangement operable to measure a fluorescence intensity, a temperature sensor and a turbidity sensor, the outputs of which are used in combination to derive a value for the BOD concentration of a sample, wherein the output of the temperature sensor is used to apply a correction to the sensed fluorescence intensity value using the equation
$${F}_{ref} = \frac{{F}_{mes}}{1 + ρ ( {T}_{mes} - {T}_{ref} )}$$
where F is the fluorescence intensity signal, T is temperature (° C.), and the subscripts mes and ref represent the measured and reference values respectively, and ρ is a temperature compensation coefficient.

2. A sensor according to claim 1, wherein the fluorescence sensor arrangement is arranged to irradiate the sample with light of wavelength in the region of 260-300 nm, and to detect fluorescence of wavelength in the region of 295-405 nm.

3. A sensor according to claim 2, wherein the fluorescence sensor arrangement is arranged to irradiate the sample with light of wavelength in the region of 285 nm, and to detect fluorescence of wavelength in the region of 350 nm.

4. A sensor according to claim 1, wherein the fluorescence sensor arrangement is arranged to irradiate the sample with light of wavelength in the region of 365 nm, and to detect fluorescence of wavelength in the region of 490 nm.

5. A sensor according to claim 1, further comprising a cleaning device operable to clean at least part of the sensor.

6. A sensor according to claim 5, wherein the cleaning device comprises a wiper.

7. A water testing method comprising the steps of using a fluorescence sensor arrangement to measure a fluorescence intensity of a sample in response to irradiation of the sample with electromagnetic radiation of an excitation wavelength, measuring the temperature and turbidity of the sample, using the measured temperature and turbidity to derive a corrected fluorescence intensity, and using the corrected fluorescence intensity to derive an indication of the BOD concentration of the sample, wherein the measured temperature is used to apply a correction to the sensed fluorescence intensity value using the equation
$${F}_{ref} = \frac{{F}_{mes}}{1 + ρ ( {T}_{mes} - {T}_{ref} )}$$
where F is the fluorescence intensity, T is temperature (° C.), and the subscripts mes and ref represent the measured and reference values respectively, and ρ is a temperature compensation coefficient.
